SEQUENCE LISTING (6 sequences)

SEQ ID NO: 1 - DNA, 18 bp, synthetic construct
  misc_feature 1..18: note = Chemically Synthesized
  source 1..18: mol_type = other DNA
gtccaaagcggcgatttg

SEQ ID NO: 2 - DNA, 20 bp, synthetic construct
  misc_feature 1..20: note = Chemically Synthesized
  source 1..20: mol_type = other DNA
ccagccatgcacactgatac

SEQ ID NO: 3 - DNA, 20 bp, synthetic construct
  misc_feature 1..20: note = Chemically Synthesized
  source 1..20: mol_type = other DNA
aagcgcataacaagcgagat

SEQ ID NO: 4 - DNA, 20 bp, synthetic construct
  misc_feature 1..20: note = Chemically Synthesized
  source 1..20: mol_type = other DNA
tgacggaatgcatttgatgt

SEQ ID NO: 5 - DNA, 20 bp, synthetic construct
  misc_feature 1..20: note = Chemically Synthesized
  source 1..20: mol_type = other DNA
ctgaaagcgtgcagtctgag

SEQ ID NO: 6 - DNA, 20 bp, synthetic construct
  misc_feature 1..20: note = Chemically Synthesized
  source 1..20: mol_type = other DNA
tgtgcgttcaaagactcgat

(19) United States
(12) Patent Application Publication
(10) Pub. No.: US 2026/0199577 A1
(43) Pub. Date: Jul. 16, 2026

(54) METHOD AND DEVICE FOR ENRICHING AND DETECTING MICROORGANISMS IN A BIOLOGICAL SAMPLE

(71) Applicant: Micronbrane Medical Co., Ltd., Taoyuan City (TW)

(72) Inventors: Yung Chang, Taoyuan City (TW); Mengchu Wu, Zhubei City (TW); Jheng-Fong Jhong, Kaohsiung City (TW); Yan-Wen Chen, Kaohsiung City (TW); Hau Hung, Zhubei City (TW)

(73) Assignee: Micronbrane Medical Co., Ltd, Taoyuan City (TW)

(21) Appl. No.: 19/631,406

(22) Filed: Mar. 27, 2026

Related U.S. Application Data
(62) Division of application No. 17/200,936, filed on Mar. 15, 2021.

(30) Foreign Application Priority Data
Mar. 27, 2020 (CN) 202010230413.4

(51) Int. Cl. A61M 1/34; B01D 39/16

(52) U.S. Cl. CPC A61M 1/3496; B01D 39/16; B01D 2239/0407; B01D 2239/0478

(57) ABSTRACT

Disclosed are a method and system for enriching and detecting microorganisms in a biological sample. The method allows the biological sample to be filtered through a polymer-modified substrate. The polymer-modified substrate is highly specific in capturing or separating human-derived nucleated cells, and allows the microorganisms to penetrate through it. During the process, a high level of microorganisms (bacteria, mycoplasmas, fungi, viruses, spores etc.) can be enriched in the sample and thus the interference from nucleated cells such as leukocytes can be reduced.

### CROSS REFERENCE TO RELATED APPLICATIONS

This application is a divisional of U.S. patent application Ser. No. 17/200,936, which was filed Mar. 15, 2021, and claims the benefit of Chinese Patent Application No. 202010230413.4, filed Mar. 27, 2020, both of which are incorporated herein by reference as if fully set forth.

The sequence listing electronically filed with this application titled “DEEMMC-PT0011_SL.xml,” which was created on Mar. 26, 2026, and has a size of 8,712 bytes, is incorporated herein by reference as if fully set forth.

### FIELD

The present invention relates to the technical field of detection of microorganisms; particularly, to a method and device for enriching and detecting microorganisms in a biological sample by reducing the interference from human-derived nucleated cells in the biological sample.

### BACKGROUND

When using molecular detection methods to detect pathogenic microorganisms (including bacteria, mycoplasmas, fungi, viruses, spores etc.) in biological samples, the cell count and genome size of human cells in the samples are much larger than the pathogenic microorganisms, and the amount of human DNAs is usually tens of thousands or even millions of times that of DNAs of pathogenic microorganisms. Therefore, during the process of molecular detection of pathogenic microorganisms, the background interference of human DNAs has always been a major challenge. Currently, there are methods for differential lysis, such as QIAamp DNA Microbiome Kit and the recently published modified method (Nanopore metagenomic enables rapid clinical diagnosis of bacterial lower respiratory infection. Charalampous et al., Nature Biotechnology, 2019); as well as methylation modification methods to remove human DNAs, such as NEBNext® Microbiome DNA Enrichment Kit (New England Biol abs). However, these methods have distinct disadvantages of complicated operations and inconsistent effects.

### SUMMARY

The present invention is to provide a method and device for enriching and detecting microorganisms in a biological sample by reducing the interference from human-derived nucleated cells such as leukocytes in the biological sample.

The present invention provides a method for enriching and detecting microorganisms in a biological sample, which includes the following steps: a) collecting the biological sample; b) filtering the sample through Sterile Acrodisc® White Blood Cell Syringe Filter (PALL) or a polymer-modified substrate, human-derived nucleated cells in the sample are captured or separated while the microorganisms in the sample pass or flow through the filter or polymer-modified substrate into filtrate; and. c) detecting the microorganisms present in the filtrate. The nucleated cells include one or more of erythroblasts, leukocytes and cancer cells. The polymer is prepared by the polymerization of one or more monomers having the structure of the formula (1):

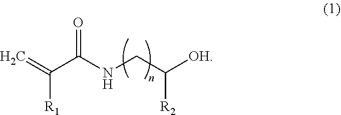

In formula (1), RI is independently selected from the group consisting of hydrogen, methyl, ethyl, hydroxyl, 01.12 alkyl, phenyl; R2 is independently selected from the group consisting of hydrogen, methyl, ethyl, C1_6 alkyl, amino, phenyl; and n is an integer of I to 5.

Preferably, the microorganisms in the biological sample are bacteria. Preferably, the microorganisms in the biological sample are fungi.

Preferably, the human-derived nucleated cells are leukocytes.

In some embodiments, the retention rate of the microorganisms in the filtrate is above 65° A.

In some embodiments, the retention rate of the microorganisms in the filtrate is above 80%.

In some embodiments, the erythrocytes can pass or flow through the polymer-modified substrate into the filtrate, and the retention rate of the erythrocytes is above 80%.

In some embodiments, the platelets can pass or flow through the polymer-modified substrate into the filtrate, and the retention rate of the platelets is above 80%.

In some embodiments, the fibrinogens can pass or flow through the polymer-modified substrate into the filtrate, and the retention rate of the fibrinogens is above 80%.

In some embodiments, the detection rate of the microorganisms in the filtrate is 2 fold higher than the samples without filtration.

In some embodiments, the detection rate of the microorganisms in the filtrate is 40 fold higher than the sample without filtration.

In some embodiments, the monomer of formula (I) comprises N-Hydroxyethyl acrylamide, N-(2-Hydroxyethyl) acrylamide, INHEMAA, and N-(2-Hydroxyethyl) acrylamide, HEAA.

In some embodiments, the polymer further comprises an additional monomer, which may be butyl methacrylate, and the monomer of formula (1) is copolymerized with described additional monomer to form a copolymer.

In some embodiments, the polymer has the structure of formula (2):

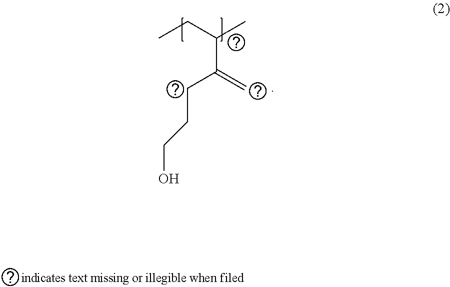

In formula (2), n is an integer of 10 to 50.

In some embodiments, the polymer has the structure of formula (4):

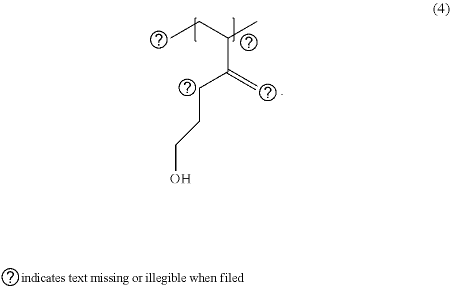

In formula (4), t is an integer of 50 to 90, n is an integer of 10 to 50, and R₂ is

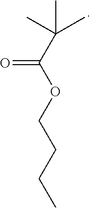

In some embodiments, the polymer is a segmented polymer.

In some embodiments, the polymer is disposed on the substrate in manners such as coating, spraying, or impregnating. The substrate includes, but is not limited to, polypropylene, polyethylene terephthalate, cellulose, polybutylene terephthalate. Elements of the surface of the modified substrate comprise carbon; oxygen; and nitrogen; the total mole percentage of carbon, oxygen, and nitrogen is defined as 100%, the mole percentage of carbon is from about 76.22% to 79.84%, the mole percentage of oxygen is from about 18.1% to 21.04%, and the mole percentage of nitrogen is from about 2.05% to about 2.75%.

In some embodiments, the filtrate is subjected to DNA purification, and is analyzed by PCR, qPCR, digital PCR, NGS, MassSpec, or Nanopore sequencing.

In some embodiments, the filtrate is subjected to DNA purification, a sequencing library is constructed by Oxford Nanopore rapid library construction process. Then, sequencing is performed with Oxford Nanopore GridiON sequencer.

In some embodiments, the biological samples is selected from the group consisting of blood, cerebral spinal fluid, cells, a cellular extract, a tissue sample, and a tissue biopsy.

In some embodiments, the method for enriching and detecting microorganisms in a biological sample according to the present invention can be used for pathogenic examination of biological samples.

In some embodiments, the invention also provides a device for enriching and detecting microorganisms in a biological sample. The device contains the following components: upper housing, filter, and lower housing. The filter is located between the upper housing and lower housing. The filter material contains the polymer-modified substrate as mentioned above; preferably, the filter is made from the polymer-modified substrate. The upper housing of the device may be provided with an inlet while the lower housing may be provided with an outlet. The biological sample enters the device from the inlet of the upper housing, penetrates through the filter, and flows out from the device through the outlet of the lower housing.

The method and device for enriching and detecting microorganisms in a biological sample provided by the present invention enable the sample to be filtered through the Sterile Acrodisc® White Blood Cell Syringe Filter (PALL) or polymer-modified substrate, which is highly specific in capturing or separating human-derived nucleated cells such as leukocytes. Besides, the microorganisms can pass through the Sterile Acrodisc® White Blood Cell Syringe Filter (PALL) or polymer-modified substrate into the filtrate, thus enriching the microorganisms (including bacteria, mycoplasmas, fungi, viruses, spores etc.) in the sample during the reduction of human-derived nucleated cells. This process can therefore reduce the interference of the human cells in pathogenic examination.

In some embodiments, the method and device for enriching and detecting microorganisms in a biological sample comprises diagnosing sepsis in the individual.

The term “capture” and grammatical variations thereof means that the human-derived nucleated cells in the sample contact with the surface of the above substrate, and are attracted by the hydrophobic interactions, hydrogen bonding, or electrostatic intermolecular forces between the substrate and the cells, causing various types of human-derived nucleated cells to adhere directly to the surface of the modified substrate. The small size cells including fibrinogens and platelets may be attached before other larger cells. These processes are defined as “capture” of human-derived nucleated cells.

The term “separation” and grammatical variations thereof refers to the separation of human-derived nucleated cells from a sample after passing the sample containing human-derived nucleated cells through the material used to separates them. It also means that the content of human-derived nucleated cells in the sample can be reduced, or even significantly reduce. The process allows the concentration of human-derived nucleated cells in the resulting filtrate to be less than the original. nucleated cells-containing sample.

The expression “reduction of nucleated cells” and grammatical variations thereof is not intended to mean that all or substantially all nucleated cells are completely removed. Instead, it is used to broadly indicate that the cell count of human-derived nucleated cells is reduced during separation or filtration.

### BRIEF DESCRIPTION OF THE DRAWINGS

FIG. 1: Schematic diagram of the process of the present invention for filtering a biological sample through a polymer-modified substrate,

FIG. 2: The filtering device of the present invention for enriching and detecting microorganisms in a biological sample,

FIG. 3: Structural formulas of the monomers and polymers of the present invention, as well as theoretically predicted values of chemical shifts of nuclear magnetic resonance (NMR) spectrum signals thereof.

FIG. 4: Measured map of -NIVilt spectra of the monomers and polymers of the present invention.

FIG. 5: Coating density results of substrates of the present invention, i.e., PP, PET, cellulose, and PBT modified with B-r-H and B-r-D.

### SYMBOL DESCRIPTION

1. Upper housing; 2. Filter; 3. Lower housing.

### DETAILED DESCRIPTION

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well-known and commonly used in the art. Known methods and techniques are generally performed according to conventional methods well known in the art and as described in various general and more specific references that are discussed throughout the present specification unless otherwise indicated. Purification and DNA sequencing techniques are performed according to manufacturer's specifications, as commonly accomplished in the art or as described herein. The nomenclatures used in connection with the laboratory procedures and techniques described herein are those well-known and commonly used in the art.

In the present invention, a biological sample can be filtered through Sterile Acrodisc’ White Blood Cell Syringe Filter (PALL) or a polymer-modified substrate. The filter and modified substrate all have a highly specific human-derived nucleated cells capture or separation ability. Also, microorganisms in the biological sample can pass through the filter or polymer-modified substrate and enters filtrate. During the reduction of human-derived nucleated cells, a high level of microorganisms (bacteria, mycoplasmas, fungi, viruses, spores etc.) can be enriched in the sample, and thus reducing the interference from human-derived nucleated cells. The biological sample with effective enrichment of microorganisms can be subjected to DNA purification. A sequencing library is constructed with an appropriate concentration of the DNA purified sample according to Oxford Nanopore rapid library construction process, and it is sequenced using Oxford Nanopore GridION sequencer. Sequencing results show that Sterile Acrodise White Blood Cell Syringe Filter (PALL) and the modified substrate can specifically remove human-derived nucleated cells and enrich microorganisms.

The Sterile Acrodisc® White Blood Cell Syringe Filter used in the present invention is provide from PALL (Catalog Nos. AP-4951 & AP-4952). The filter is a proven filtration device designed to separate leukocytes from whole blood samples while allowing red blood cells (RBCs) and platelets to flow through the membrane,

The method of enriching and detecting microorganisms in a biological sample according to the present invention includes: first, obtaining the biological sample, such as blood. Secondly, filtering the sample through a polymer-modified substrate. Human-derived nucleated cells in the sample are captured or separated. The content of nucleated cells in the obtained filtrate is greatly reduced. The microorganisms can be enriched in the filtrate by using the modified substrate during the reduction of human-derived nucleated cells.

The polymer of the present invention is prepared by the polymerization of one or more monomers having the structure of the formula (1):

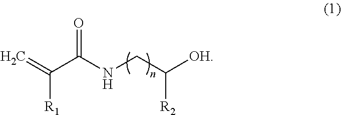

In formula (1), Rₜ is independently selected from the group consisting of hydrogen, methyl, ethyl, hydroxyl, C₁₋₁₂ alkyl, and phenyl; R₂ is independently selected from the group consisting of hydrogen, methyl, ethyl, C₁₋₆alkyl, amino, and phenyl; and n is an integer of I to 5.

According to some embodiment of the present invention, the R₁ in formula (1) is hydrogen, R₂ is hydrogen, and n is an integer of 1.

According to some embodiments of the present invention, the amide group and hydroxyl group-containing, monomer is N-Hydroxyethyl acrylamide or N-(2-hydroxyethyl) acrylamide.

According to some embodiments of the present invention, the polymer is a copolymer prepared by the copolymerization of the monomer of formula (1) and at least one additional monomer, and the additional monomer is butyl methacrylate (BMA).

According to some embodiments of the present invention, the polymer is a segmented polymer.

According to some embodiments of the present invention, the polymer prepared by the monomer of formula (1) may have the structure of formula (2):

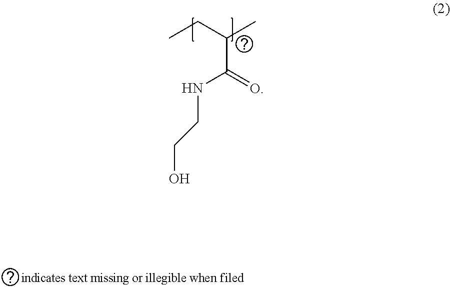

In formula (2), n is an integer of 10 to 50.

According to some embodiments of the present invention, the polymer prepared by the monomer of formula (1) may also have the structure of formula (4):

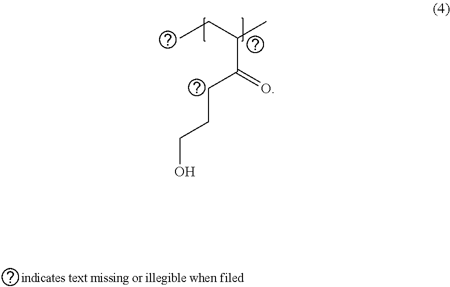

In formula (4), t is an integer of 50 to 90, n is an integer of 10 to 50.

R2 is

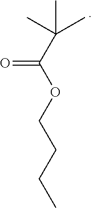

The preparation of the polymer is carried out by known technologies. For example, one monomer is used as the base end of the substrate, and another monomer is used as the functional end. The two monomers are mixed in a certain proportion, and follows with the addition of initiator ACVA. The solvent used for the preparation is ethanol. Next, the polymerization reaction is carried out at 70° C., thereby giving the desired polymer. After the reaction is completed, the product is precipitated using deionized water as precipitating agent and then dried.

The polymer of the present invention can be coated, sprayed, or impregnated on the substrate to achieve the purpose of modifying the substrate. Specifically, dissolve an amount of polymer in ethanol as solvent to prepare a polymer solution; select a suitable substrate, cut it to a suitable size, and soak it in the polymer solution for about 1 minute; and then, wash the substrate surface with deionized water and dry. Accordingly, a surface-modified substrate coated with the polymer can be obtained. Materials used to manufacture the substrate can be polypropylene (PP), polyethylene terephthalate (PET), cellulose, polvbutylene terephthalate (PBT), etc. Surface elements of the modified substrate include carbon, oxygen, and nitrogen, while the total mole percentage of them is defined as 100%. The mole percentage of carbon is about 76.22% to 79.84%, the mole percentage of oxygen is about 18.1% to 21.04%, and the mole percentage of nitrogen is about 2.05% to 2.75%.

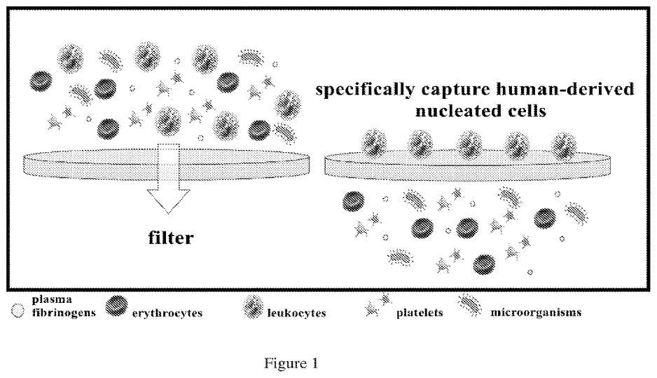

The biological sample is filtered through the polymer-modified substrate of the present invention. Refer to FIG. 1, which is a schematic diagram of the process of the present invention for filtering a biological sample through a modified substrate. The modified substrate can capture and absorb, attach or adhere human-derived nucleated cells (such as leukocytes), thus separating them from the sample such as whole blood. More importantly, the plasma proteins are hardly absorbed during the process of filtration while the platelet adhesion hardly occurs, which improves the retention rate of platelets. Besides, the rest of the biological sample (e.g., blood), such as erythrocytes, platelets, bacteria, viruses, and spores can pass/flow through the modified substrate to achieve the purification effect. The test results show that the removal rate of leukocytes in the filtered sample is greater than 70% and can even reach more than 90%. The microbial detection rate of the sample after filtration is at least twice that of the sample before filtration, and it can even reach a higher multiple, such as 40 folds.

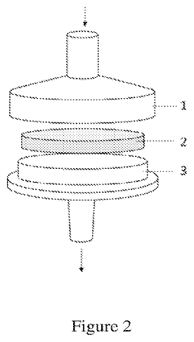

The invention also provides a filtering device matched with the polymer modified substrate. As shown in FIG. 2, the device includes an upper housing 1, a filter 2, a lower housing 3. The filter 2 is located between the upper housing 1 and the lower housing 3. A sample inlet is provided on the upper housing 1, and a sample outlet is provided on the lower housing 3. The sample enters from the inlet of the upper housing 1 and penetrates through the filter 2. After that, it flows out through the outlet of the lower housing 3. The filter 2 is prepared from the polymer modified substrate described above.

The method for enriching and detecting microorganisms of the present invention can be applied to the filtering device. First, a biological sample such as blood is obtained; second, the sample is introduced from the inlet of the upper housing 1, filtered through the filter 2, and then flowed out through the lower housing 3. The human-derived nucleated cells such as leukocytes in the sample are captured or separated, and the content of nucleated cells in the filtrate is greatly reduced. The process enables to reduce the human-derived nucleated cells from the filtrate while allowing microorganisms to pass or flow through the filter 2, In addition, this process also enriches the microorganisms in the filtrate and reduces human-derived nucleated cells inference. The filtrate with effective enriched microorganisms is subjected to the process of DNA purification. DNA purified sample is used at an appropriate concentration, a sequencing library is constructed according to Oxford Nanopore rapid library construction process, and Oxford Nanopore GridION sequencer is used for sequencing. The sequencing result shows that the modified substrate can specifically remove human-derived nucleated cells' interference and achieve the goal of enrichment of microorganisms, leading to a great increase in the proportion of microorganisms.

The determination of DNA in the present invention can be performed by common methods including PCR, qPCR, digital PCR, NGS, MassSpec, or Nanopore sequencing.

Oxford Nanopore sequencing technology used in Nanopore sequencing is the third generation single-molecule sequencing technology which has the advantages of simple sample handling, fast and long sequencing length (>10 kbp) compared with NGS second-generation technology that requires amplified signals. These advantages are very suitable for the rapid identification of clinically unknown microbial pathogens. Therefore, this method can be applied to examine sepsis, detect pathogens and so on.

The present invention is further clarified below with reference to figures and embodiments. It should be noted that the following embodiments provided are only for illustrative purpose and not intended to limit the invention. After reading the present invention, for variations obvious to one skilled in the art will be included within the invention defined by the claims. While the preferred embodiments of the invention have been illustrated and described, it will be appreciated that various changes can be made therein without departing from the spirit and scope of the invention.

### Experimental Methods and Materials

### Cell Lines

The all cell lines were obtained from American Type Culture Collection (ATCC). The TFI (ATCC© CRL-2003™) and Jurkat clone E6-1 (ATCC® 71, 1B-152™) were cultured in RPMI medium with 10% FBS, PC-3 (ATCC® CRLI435™) was cultured in F12K medium with 10% FBS. SK-BR-3 (ATTC® HTB30™) was cultured in McCoy's 5A (modified) medium with 10% FBS, and K-562 (ATCC® CCL243™) was cultured in IMDM (Iscove's Modified Dulbecco's Medium) with 10% FBS in incubator at 37° C., 5% CO₂.

### 1. Experimental Materials for Substrate Modification

The material of inventive monomer is N-Hydroxyethyl acrylamide (HEAA), which has both a hydroxyl functional group and an amide functional group, and has a chemical structure as follows:

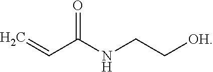

The material of comparative monomer is positively charged N,N-dimethylaminoethyl methacrylate (DMAEMA), which has the following chemical structure:

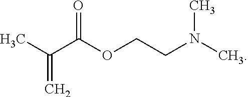

Besides, butyl methacrylate (BMA) is used as the base end of the polymer of the present invention, so that the polymer can be physically absorbed on the surface of substrate. BMA has the following chemical structure:

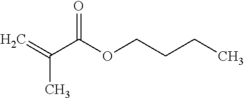

4,4′-azobis (4-cyanovaleric acid) (ACVA) is used as initiator, and has the following chemical structure:

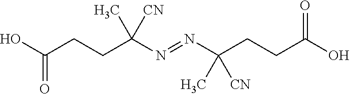 [SMILES: CC(C#N)(CCC(=O)O)/N=N/C(C)(C#N)CCC(=O)O]

The materials of the substrate include polypropylene (PP), polyethylene terephthalate (PET), cellulose, polybutylene terephthalate (PBT). The chemical structures are respectively as follows:

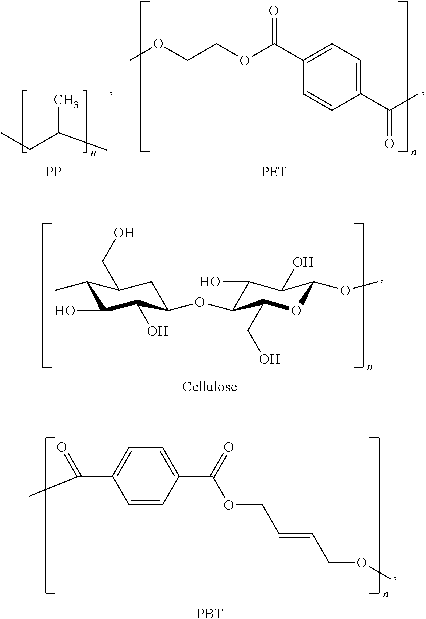

### 2. Preparation of Polymers for Substrate Modification

BMA as the base end and HEAA or DMAFMA as the functional end were mixed according to the given proportion (about 70% of the base end and about 30% of the functional end), and were added ACVA as initiator, and ethanol as solvent. Next, the polymerization reaction was carried out at 70° C. for 24 hours. BMA-r-HEAA and BMA-r-DMAEMA polymers could be prepared respectively. After the reaction was completed, the product was precipitated using deionized water as precipitating agent and then dried.

### 3. Surface Modification of the Substrate

Each of PP, PET, cellulose, and PBT was selected as the substrate to be modified.

### 3.1 PP Substrate Modification by Physical Adsorption

BMA-r-HEAA polymer and BMA-r-DMAEMA polymer were each taken and formulated into a polymer solution using ethanol as a solvent. A PP substrate was taken and cut to a suitable size, soaked in the BMA-r-HEAA polymer solution for I. minute, then the residual solution on the surface was washed off with deionized water, and then the PP substrate was dried; and similar operations as described above were done starting with a PP substrate and the BMA-r-DMAEMA polymer solution. Accordingly, surface-modified PP substrate coated with BMA-r-HEAA polymer and surface-modified PP substrate coated with BMA-r-DMAEMA polymer can be obtained respectively.

### 3.2 PET Substrate Modification by Physical Adsorption

BMA-r-HEAA polymer and BMA-r-DMAEMA polymer were each taken and formulated into a polymer solution using ethanol as a solvent. A PET substrate was taken and cut to a suitable size, soaked in the BMA-r-HEAA polymer solution for 1 minute, then the residual solution on the surface was washed off with deionized water, and then the PET substrate was dried; and similar operations as described above were done starting with a PET substrate and the BMA-r-DMAEMA polymer solution. Accordingly, surface-modified PET substrate coated with BMA-r-FIEAA polymer and surface-modified PET substrate coated with BMA-r-DMAEMA polymer can be obtained respectively.

### 3.3 Cellulose Substrate Modification by Physical Adsorption

BMA-r-HEAA polymer and BMA-r-DMAEMA polymer were each taken and formulated into a polymer solution using ethanol as a solvent. A cellulose substrate was taken and cut to a suitable size, soaked in the BMA-r-HEAA polymer solution for 1 minute, then the residual solution on the surface was washed off with deionized water, and then the cellulose substrate was dried; and similar operations as described above were done starting with a cellulose substrate and the BMA-r-DrvlAErvlA polymer solution. Accordingly, surface-modified cellulose substrate coated with BMA-r-BEAA polymer and surface-modified cellulose substrate coated with BMA-r-DMAEMA polymer can be obtained respectively.

### 3.4 PBT Substrate Modification by Physical Adsorption

BMA-r-HEAA polymer and BMA-r-DMAEMA polymer were each taken and formulated into a polymer solution using ethanol as a solvent. A PBT substrate was taken and cut to a suitable size, soaked in the BMA-r-HEAA polymer solution for 1 minute, then the residual solution on the surface was washed off with deionized water, and then the PBT substrate was dried; and similar operations as described above were done starting with a PBT substrate and the BMA-r-DMAEMA polymer solution. Accordingly, surface-modified PBT substrate coated with BMA-r-T-MA A polymer and surface-modified PBT substrate coated with BMA-r-DMAEMA polymer can be obtained respectively.

### 3.5 Identification of Nuclear Magnetic Resonance (NMR)

10 mg of each of the above polymers was weighted and dissolved in 1 mL of methanol (d-MeOH) to give a solution with a concentration of 10 mg/mL, respectively. The solution was placed in an NMR test tubes, and delivered to the Instrumentation Center of National Central University in Taiwan for performing measurements. Then the chemical structures and the monomer ratios of the polymers were analyzed and calculated by the characteristic peaks of the spectra.

### 3.6 Density Measurements of Surface Modification

Before modification, the PP, PET, cellulose, and PBT substrates were weighted by a microbalance. After the surfaces were modified completely, the substrates were dried, and the weights of the surface-modified substrates were measured via a microbalance. The weight of the polymer modified on the surface of the substate, in each case, can he obtained by calculating the difference in weights of the substrate before and after the modification. Finally, the weight of the polymer per unit area, i.e., the surface coating density, can be obtained through conversion.

### 3.7 Blood Filtration Test

The modified PP, PET, cellulose, and PBT substrates were each cut into a circle with a diameter of 2.6 cm, and 20 layers of the substrates were staked. Then, the substrates were placed and locked in a filtering device (similar to the device shown in FIG. 2). This part of the experiment is divided into two methods:
Method 1: 10 mL of whole blood was filtered through the modified substrate in the filter. Then, the blood sample before and after the filtration were examined using hemocytometer to calculate the leukocyte removal rate and platelet retention rate.
Method 2: 1 mL of E. coli liquid (6×10⁹ cells/mL) was added to 9 mL of blood sample, and shaken for 5 minutes in a blood test tube mixer. Then, the blood sample was taken out and filtrated. Mixtures containing blood sample and other bacteria (such as Staphylococcus aureus) or fungi (such as Aspergillus brasiliense) were also prepared in the same way.

### 3.8 Enzyme Linked Immunosorbent Assay (ELISA)

The blood samples obtained before and after the filtration of method 1 and method 2 from embodiment 3.7 were centrifuged using a centrifuge respectively. The supernatant (i.e., plasma) was extracted and diluted 10-fold with PBS. After dilution, 0.25 mL of the sample was taken out and transferred to a 24-well plate (24 well-tissue culture polystyrene plate, 24-well TCPS plate). To the sample was added 0.25 mL of the first antibody specific for Fibrinogen (Monoclonal Anti-human Fibrinogen, Clone, Sigma_Aldrich Co., cat: F4639), and placed in oven at 37° C. for 30 minutes. 0.25 int, of secondary antibody (anti-mouse IgG, rabbit IgG whole, I-IRP conjugated, Wako Co., cat: 014-1761) was added, which is specific for the first antibody and will specifically bind to the first antibody. After being placed in a 37° C. oven for 30 minutes, 0.25 mL of a developer (3,3′,5,5′-Tetramethylbenzidine, TMB) was added, waiting 6 minutes for color development, and then 0.25 mL of 1 M sulfuric acid was added to each sample to stop the reaction. 200 μl of solution from each sample (including TCPS empty wells) was pipetted into a 96-well plate and analyzed at a UV wavelength of 450 nm by using Bio-tek model PowerWare XS microplate reader, After obtaining the UV readings for the samples before and after filtration, the protein recovery ratio can be obtained.

### 3.9 Microbial Filtration Test

Use a variety of different microorganisms, including fungi and Gram (−) and Gram (+) bacteria, such as Aspergillus brasiliensis, E. coli, Shewanella algae (SA), Staphylococcus pseudintermedius (SP), Staphylococcus aureus, Klebsiella pneumoniae (CRKP), Imtechella halotolerans (I. Halo), and Allobacillus halotolerans (A. Halo) etc. The same amount of microbial liquid was filtered or not filtered. The Sterile Acrodisc® White Blood Cell Syringe Filter (PALL), modified PP, PET, cellulose, and PBT substrates were used to test the capacity of microbial filtration.
Group 1: Shewanella algae (Gram−), Staphylococcus pseudintermedius (Gram−), and Klebsiella pneumoniae (Gram−).
Group 2: E. coli (Gram−), Staphylococcus aureus (Gram+), and Aspergillus brasiliensis (fungi).
Group 3: Imtechella halotolerans (Gram−) and Allobacillus halotolerans (Gram+).

### 3.10 DNA Purification, Concentration, and Quantitative Sequencing

The blood samples added with bacteria prepared under method 2 of embodiment 3.7 were divided into two, one was filtered, and the other was not filtered as a control. The samples were each centrifuged and the supernatant (i.e. plasma) was extracted. The supernatants and bacterial filtration samples of embodiment 3.9 were each subjected to DNA purification by DNeasy Blood and Tissue Kit. The concentration of purified DNA was measured by nanodrop spectrophotometer for comparison of the concentrations before and after filtration, and analyzed for the corresponding bacteria or fungi by Semi-quantitative Real-Time PCR. An appropriate concentration of DNA. was taken and a sequencing library was constructed according to the Oxford Nanopore rapid library construction process. The sequencing results of Oxford Nanopore GridION sequencing proves that the modified substrates remove the host leukocytes and human DNAs, and enrich the microbial DNAs in the samples.

### 4. The Detection Results of Substrate Modification

### 4.1 Analysis of Nuclear Magnetic Resonance (NMR)

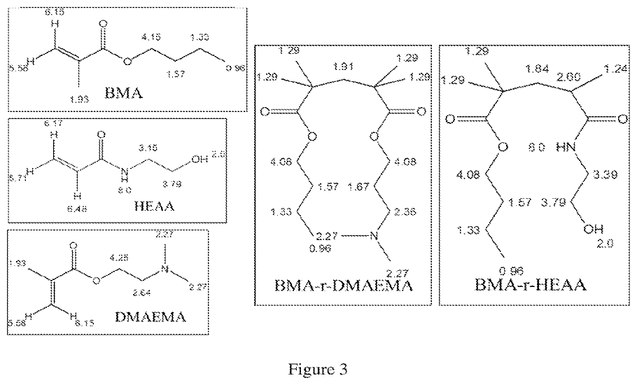

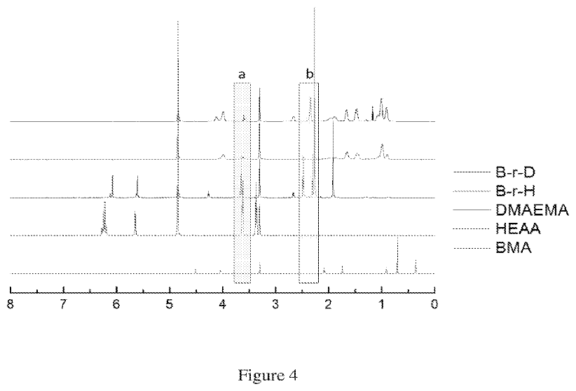

As shown in Table 1 below, for the convenience of further description, the polymer compounds such as BMA-r-HE.AA. and BMA.-r-DMAEMA are abbreviated as B-r-H and B-r-D respectively. FIG. 3 shows the structural formulas and their theoretically predicted values of chemical shift of each one of the monomer compounds and polymer compounds. FIG. 4 shows the measured map of NMR spectra of monomer and polymer structures. ¹H NMR (300 MHz, DMSO-d₆) δ 13.21 (s, 1H), 10.25 (s, 1H), 9.10 (s, 1H), 8.66 (s, 1H), 8.25 (s, 1H), 8.12 (s, 1H), 8.10-7.82 (m, 5H), 7.17 (d, J=8.7 Hz, In), 7.14 (t, =74.7 Hz, 1H), 3.77-3.73 (m, 4H), 3.04-3.76 (m, 4H). The specific experimental conditions and actual test values are listed in Table 1. According to the NMR analysis, the characteristic peak of HEAA is mainly appeared at site “a”, and the characteristic peak of DMAFMA is mainly appeared at site “b”. It is shown according to the NMR spectra that the polymer compounds used in this experiment have been successfully synthesized.

TABLE 1

| Polymer/monomer name | Abbreviation | NMR test condition and data |
|---|---|---|
| BMA-r-HEAA | B-r-H | ¹H NMR (600 MHz, Methanol-d₄) δ 3.99 (s, 2H), 3.61 (s, 1H), 1.97 (s, |
| | | 2H), 1.66 (s, 3H), 1.46 (s, 3H), 0.99 (s. 6H) 0.90 (s, 1H). |
| BMA-r-DMA | B-r-D | ¹H NMR. (600 MHz, Methanol-d₄) δ 4.12 (s, 1H), 3.99 (s, 2H), 2.66 |
| | | (s, 1H), 2.35 (d, J = 10.3 Hz., 3H), 1.99-1.94(m, 2H), 1.90-1.86 |
| | | (m, 1H), 1.67 (s, 3H), 1.48 (s, 3H), 1.08 (s, 2H), 1.01 (s, 4H), 0.91 (s, |
| | | 3H). |
| DMAEMA | DMAEMA | ¹H NMR (600 MHz, Methanol-d₄) δ 6.07 (dt, J = 2.4, 1.2 Hz, |
| | | 211), 5.64-5..58 (m, 2H), 4.84 (d, J = 13.5 Hz, 1H), 3.65 (td, J = |
| | | 6.2, 2.0 Hz, 4H), 2.48 (t, J = 6.1 Hz, 4H), 2.33-2.24 (m, 5H), |
| | | 2.27 (s, 10H), 1.93 (dt, J = 7.4, 1.4 Hz, 7H). |
| HEAA | HEAA | ¹H NMR (600 MHz Methanol- d₄) δ 6.07 (s, 1H), 5.60 (q, J = 1.7 Hz, |
| | | 1H), 4.15 (td, J = 6.6, 1.4 Hz, 2H), 1.92 (s, 3H), 1.70-1.62 (m, 2H), |
| | | 1.47-1 .38 (m, 2H), 0.96 (td, J = 7.4, 1.3 Hz, 3H). |
| BMA | BMA | ¹H NMR (600 MHz, Methanol- d₄) δ 6.30-6.17 (m, 2H), 5.64 |
| | | (dt, J = 9.9, 2.1 Hz, 1H), 3.62 (td, J = 5.8, 1.7 Hz, 2H), 3.37 |
| | | (td, J = 5.8, 1.8 Hz, 2H). |

### 4.2 Coating Density Measurement Results of PP, PET, Cellulose, and PBT Substrates

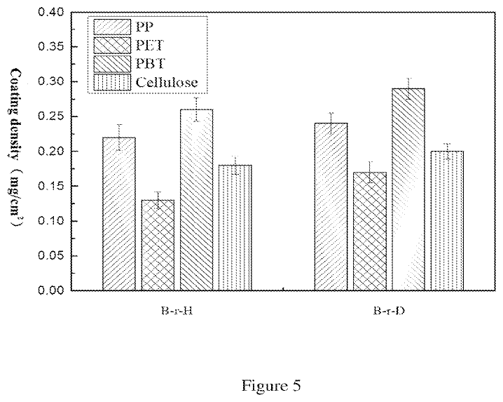

FIG. 5 shows the coating density results of PP, PET, cellulose, and PBT substrates each modified with any of B-r-H and B-r-D, By respectively weighing the weights of the substrates before and after modification as well as measuring the surface areas of the substrates, the coating density of each polymer on the surface of each of the substrates can be calculated, and the densities were ranging from about 0.1 to about 0.3 mg/cm².

### 4.3 Filtration Test Results of Blood Cells

The modified substrate is used to filter blood as well as blood added with bacteria as described for blood filtration test above. The content of various blood cells before and after filtering in the blood samples can be determined by a hemocytometer. Accordingly, the leukocyte removal rate and the retention rate of each of various blood cells can be calculated. Fibrinogen was detected by ELISA, and bacteria such as E. Coll are detected using an absorption spectrometer.

RBC is referred to red blood cell (erythrocyte), WBC is referred to white blood cell (leukocyte), PLT is referred to platelet, E. coli is referred to as Escherichia coli, and Fibrinogen is referred to plasma fibrinogen. As shown in Table 2, the substrates modified by HEA A have a leukocyte capture rate of at least 94%, and retains more than 93% of erythrocytes, more than 87% of platelets, more than 93% of plasma fibrinogens. Thus, when whole blood flows through the modified substrate, most of the leukocytes will adhere to the substrate while most of the erythrocytes, platelets, and plasma fibrinogens can be retained in the filtrate. Accordingly, the B-r-H modified substrate of the present invention is high specific in capturing leukocytes without capturing or attaching erythrocytes and platelets. Thus, it is considered as a good material for leukocyte reduction for whole blood sample. Certainly, in other embodiments, for instance, an erythrocyte concentrate or platelet concentrate can also be allowed to penetrate through the above-mentioned modified substrate to specifically remove leukocytes.

The method of capturing, separating, or filtering leukocytes using substrate modified by B-r-H not only increases the capture rate of leukocytes, but also retains most of the erythrocytes, platelets, and plasma fibrinogens. Therefore, it is obvious that the method of filtering blood through substrate modified by B-r-H is effected by a different mechanism of blood filtration. compared to the method of filtering leukocytes through substrate modified by polymer such as B-r-D and D'IAEMA.

Table 3 shows the result of filtration after adding E. Coll to the blood. The result shows that the HEAA-modified substrate has a leukocyte capture rate of at least 95%, and retains more than 92% of the erythrocytes, more than 87% of platelets. Besides, the retention rate of the E. Coli is more than 82%. This means that when whole blood flows through the modified substrate, most of the leukocytes will adhere to the substrate, and the remaining filtrate can retain other cells and bacteria in the blood. Accordingly, the B-r-H modified substrate of the present invention has a very high specificity for leukocyte capture compared to the conventional method of capturing, separating, or filtering leukocytes through substrate modified with polymers such as B-r-D and DMAEMA. In addition, the B-r-H modified substrate will not capture or attach erythrocytes and platelets. It can retain most of the erythrocytes, platelets, and plasma fibrinogens in the filtrate, and most of the bacteria can pass through the modified substrate without attachment. Thus, when using the filtrate with leukocytes specifically removed, the accuracy and efficiency of pathogenic examination can be effectively improved.

TABLE 2

| | | | | | RBC | WBC | PLT | Fibrinogen |
|---|---|---|---|---|---|---|---|---|
| | | RBC | WBC | PLT | retention | removal | retention | retention |
| | | (10⁶/uL) | (10³/ uL) | (10³/ uL) | rate | rate | rate | rate |
| before filtration | | 3.75 | 6.54 | 263 | — | — | — | 100.00% |
| polymer | substrate | | | | | | | |
| B-r-H | PP | 3.53 | 0.27 | 231 | 94.13% | 95.87% | 87.83% | 93.06% |
| | PET | 3.54 | 0.25 | 229 | 94.40% | 96.18% | 87.07% | 96.07% |
| | PBT | 3.52 | 0.35 | 233 | 93.87% | 94.65% | 88.59% | 94.95% |
| | Cellulose | 3.55 | 0.32 | 235 | 94.67% | 95.11% | 89.35% | 96.31% |
| B-r-D | PP | 3.46 | 0.16 | 111 | 92.27% | 97.55% | 42.21% | 45.84% |
| | PET | 3.47 | 0.24 | 119 | 92.53% | 96.33% | 45.25% | 47.90% |
| | PBT | 3.46 | 0.23 | 114 | 92.27% | 96.48% | 43.35% | 46.39% |
| | Cellulose | 3.44 | 0.27 | 109 | 91.73% | 95.87% | 41.44% | 50.75% |

TABLE 3

| | | | | | RBC | WBC | PLT | | E. coil | Fibrinogen |
|---|---|---|---|---|---|---|---|---|---|---|
| | | RBC | WBC | PLT | Retention | removal | retention | E. coil | retention | retention |
| | | (10⁶/uL) | (10³/uL) | (10³/uL) | rate | rate | rate | OD600 | rate | rate |
| before filtration | | 3.47 | 6.20 | 221 | — | — | — | 0.784 | — | 100.00% |
| polymer | substrate | | | | | | | | | |
| B-r-H | PP | 3.25 | 0.31 | 194 | 93.66% | 95.00% | 87.78% | 0.667 | 85.08% | 95.71% |
| | PET | 3.29 | 0.23 | 196 | 94.81% | 96.29% | 88.69% | 0.650 | 82.91% | 96.72% |
| | PBT | 3.20 | 0.30 | 193 | 92.22% | 95.16% | 87.33% | 0.656 | 83.67% | 95.16% |
| | Cellulose | 3.26 | 0.25 | 198 | 93.95% | 95.97% | 89.59% | 0.659 | 84.06% | 94.06% |
| B-r-D | PP | 3.25 | 0.31 | 100 | 93.66% | 95.00% | 45.25% | 0.254 | 32.40% | 46.43% |
| | PET | 3.26 | 0.30 | 99 | 93.95% | 95.16% | 44.80% | 0.241 | 30.74% | 49.39% |
| | PBT | 3.23 | 0.32 | 101 | 93.08% | 94.84% | 45.70% | 0.272 | 34.69% | 45.44% |
| | Cellulose | 3.27 | 0.28 | 97 | 94.24% | 95.48% | 43.89% | 0.254 | 32.40% | 48.16% |

### 4.4 DNA Concentration Results of Bacterial Filtration Test

According to the bacterial filtration test method mentioned above, group I. of bacteria were subjected to filtration test using B-r-H modified substrate, which is highly specific for leukocytes capture and can retain other cells as well as bacteria. Table 4 shows data of the identical amount of bacterial solution, including Shewanella algae (SA), Staphylococcus pseudintermedius (SP), Klebsiella pneumoniae (CRKP). After filtering through B-r-11 modified substrate, the amount of DNA recovered in the filtered sample can reach more than 92% of the sample without filtering. This result indicates that most of the common bacteria are allowed to penetrate through the highly specific modified substrate.

TABLE 4

| | Filtrate Vol. | | DNA conc. (ng/ul) | DNA conc. (ng/ul) | DNA |
|---|---|---|---|---|---|
| Bacteria ID | (ul) % of V Loss | | from 1 ml filtrate | from 1 ml control | Recovery (%) |
| SA (Gram−) | 1730 | 13.5 | 3.76 | 4.07 | 92.38 |
| SP (Gram+) | 1640 | 18.0 | 1.86 | 1.7 | 109.41 |
| CRKP (Gram−) | 1670 | 16.5 | 3.07 | 2.82 | 108.87 |

### 4.5 Semi-Quantitative Real-Time PCR Semi-Quantitative Comparison Results of Blood Simulation Samples

Group 2 of microorganisms were subjected to the test. 10⁶ CF1J/mL or 10⁴ CFU/mL of mixed microorganisms (E. coli:Staphylococcus aureus:Aspergillus brasiliensis=1:1:1) was added to normal human whole blood. 5 mL of the sample was centrifuged at 2,000 g for 15 mins, the upper plasma was removed and filtered with a B-r-H modified substrate or unfiltered. The sample was then centrifuged at 13,500 rpm for 10 mins to remove the supernatant and DNA of the bottom product was purified. The purified DNAs were subjected to a semi-quantitative comparison using primers (table 5) of specific bacteria, according to SYBR-green Semi-quantitative Real-Time PCR semi-quantitative method on Light Cycler 96 (Roche) PCR instrument. The semi-quantitative analysis results were shown in Table 6. W/O filter is referred to unfiltered samples, while W/filter is referred to filtered samples. The corresponding concentrations of E. coli (Gram−), Staphylococcus aureus (Gram ±), and Aspergillus brasiliensis (fungi) in blood samples filtered through B-r-H modified substrates and in unfiltered blood samples were not significantly different. The results show that after filtering through B-r-1-1 modified substrate, most of the bacteria and fungi in the blood samples can penetrate through this substrate highly specific in leukocyte capture.

TABLE 5

| SEQ | | |
|---|---|---|
| ID No. | Primer Name | Primer sequence |
| 1 | E. coli-F | GTCCAAAGCGGCGATTIG |
| 2 | E. coli-R | CCAGCCA.TGCACACTGATAC |
| 3 | S. aureus-F | AAGCGCATAACAAGCGAGAT |
| 4 | S. aureus-R | TGACGGAATGCATTTG.ATGT |
| 5 | A. | CTGAAAGCGTGCAGTCTGAG |
| | brasiliensis_18S-F | |
| 6 | A. | TGTGCG11CAAAGACTCGAT |
| | brasiliensis 18S-R | |

TABLE 6

| | w/o filter | | | w/filter | | | |
|---|---|---|---|---|---|---|---|
| Bacteria/Fungi | Ct1 | Ct2 | Average Ct | Ct1 | Ct2 | Average Ct | ΔCt |
| 10\hat{ }6 E. Coli (Gram−) | 23.6 | 23.9 | 23.7 | 24.3 | 24.3 | 24.3 | 0.6 |
| 10\hat{ }4 E. Coli (Gram−) | 30.9 | 30.9 | 30.9 | 30.8 | 31.1 | 31.0 | 0.1 |
| 10\hat{ }6 S. aureus (Gram+) | 31.5 | 31.6 | 31.6 | 30.9 | 31.1 | 31.0 | −0.6 |
| 10\hat{ }4 S. aureus (Gram+) | 35.8 | 35.9 | 35.9 | 36.1 | 36.1 | 36.1 | 0.3 |
| 10\hat{ }6 A. brasiliensis | 23.3 | 23.5 | 23.4 | 23.9 | 24.2 | 24.0 | 0.6 |
| 10\hat{ }4 A. brasiliensis | 30.8 | 31.0 | 30.9 | 31.1 | 30.6 | 30.8 | −0.1 |

### 4.6 Metagenomic Sequencing Results of Blood Simulation Samples

### 4.6.1 In Situ Positive Control Test:

ZyrnoBIOMICS™ Spike-in Control I (High Microbial Load) (ZYMO RESEARCH, Catalog Nos. D6320 & D6320-1.0) was used as an in situ positive control, which consists of equal cell numbers of two bacteria strains, Imtechella halotolerans and Allobacillus halotolerans. Normal human whole blood samples were added with 1×10⁴ and 1×10⁶ cells/mL of total bacteria, respectively. 5 mL of the sample was centrifuged at 2,000 g for 15 mins, the upper plasma was removed and filtered with Sterile Acrodisc® White Blood Cell Syringe Filter (PALL), B-r-H modified substrate, or unfiltered, respectively. Then, the sample was centrifuged at 13,500 rpm for 10 mins to remove the supernatant and DNA in the bottom product was purified by TANBead© Nucleic Acid Extraction kit Blood Bacterial DNA Auto Plate (TAN Bead, CatM6BGA45). A sequencing library was constructed by Oxford Nanopore Technologies (ONT) SQK-RBK004 Rapid Barcoding Kit, and the purified DNAs were sequenced using ONT FLOMIN106 (revD) flow cell. The sequence results were shown in Table 7. W/O is referred to unfiltered samples, and W/f Devin and W/f PALL are referred to B-r-H modified substrate and Sterile Acrodisc® White Blood Cell Syringe Filter (PALL) filtered samples, respectively. AA and AI refer to that when the human cell is the basic value, whether the relatively amount of bacteria is greater than human cell. As the AA and AI value is lower, means the more the relatively amount of bacteria. The results show that after filtering through Sterile Acrodisc® White Blood Cell Syringe Filter (PALL) or B-r-H modified substrate, it can indeed enrich the microorganisms in the blood samples. Among them, the testing group 1×10⁴ cells/mL has the most significant effect. The concentration of pathogenic microorganisms in the general human body is also quite low, so the result is in line with our expectations.

TABLE 7

| | | | | | | |
|---|---|---|---|---|---|---|
| I. Halo | A. Halo | Homo | ΔI | ΔΔI | ΔA | ΔΔA |

1 × 10⁴ cells/mL

| | | | | | | | |
|---|---|---|---|---|---|---|---|
| w/o | 27.6 | 26.22 | 28.58 | −0.98 | — | −2.36 | — |
| w/f | 30.05 | 26.46 | 33.47 | −3.42 | −2.44 | −7.01 | −4.65 |
| Devin | | | | | | | |
| w/f | 28.06 | 26.12 | 33.04 | −4.98 | −4.00 | -6.92 | −4.56 |
| PALL | | | | | | | |

1 × 10⁶ cells/mL

| | | | | | | | |
|---|---|---|---|---|---|---|---|
| w/o | 20.23 | 18.66 | 28.99 | −8.76 | — | 10.33 | — |
| w/f | 21.4 | 20.49 | 31.1 | −9.70 | −0.94 | −10.61 | −0.28 |
| Devin | | | | | | | |
| w/f | 21.22 | 19.19 | 31.16 | −9.94 | −1.18 | −11.97 | −1.64 |
| PALL | | | | | | | |

### 4.6.2 Pathogenic Microorganisms Test:

Normal human whole blood was added with 2.5×10³ CFU/mL of E. coli and 1 ng/mL of Klebsiella pneumonia gDNA. 5 mL, of the sample was centrifuged at 2,000 g for 15 mins, the upper plasma was removed and filtered with a B-r-H modified substrate or unfiltered. Then, the sample was centrifuged at 13,500 rpm for 10 mins to remove the supernatant and DNA of the bottom product was purified. A sequencing library was constructed by Oxford Nanopore Technologies (ONT) SQK-RBK004 Rapid Barcoding Kit, and the purified DNAs were sequenced using ONT FLOMIN 106 (revD) flow cell. The sequence results were shown in Table 8. W/O filter-1 and W/O filter-2 are referred to unfiltered samples, and W/ filter-1 and W/ filter-2 are referred to filtered samples. The percentages of K. pneumonia in the samples filtered through B-r-H modified substrate increased from 0.2% and 0.3% to 10.6% and 13.4% compared to unfiltered samples, respectively. The percentages of E. coli increased from 0.05% and 0.03% to 2.8% and 5.2%, respectively. The results show that after filtering through B-r-H modified substrate, it can indeed enrich the microorganisms in the blood samples and increase the detection rate of microorganisms by more than 40 folds.

TABLE 8

| Sample | Filter | Barcode | Total Reads | Classified | Homo (%) | K. Pneumonia (%) | E. Coli (%) |
|---|---|---|---|---|---|---|---|
| Spike-in | w/filter-1 | RB01 | 4,001 | 615 | 522 (84.9%) | 65 (10.6%) | 17 (2.8%) |
| Spike-in | w/filter-2 | RB02 | 1,620 | 97 | 74 (76.3%) | 13 (13.4%) | 5 (5.2%) |
| Spike-in | w/o filter-1 | RB06 | 37,394 | 32,512 | 32,376 (99.6%) | 67 (0.2%) | 17 (0.05%) |
| Spike-in | w/o filter-2 | RB07 | 34,894 | 21,890 | 21,775 (99.5%) | 72 (0.3%) | 6 (0.03%) |
| No Spike-in | Old filter | RB08 | 1,958 | 114 | 102 (89.5%) | 8 (7.0%) | |
| blank | | RB09 | 4,061 | 12 | 6 (50.0%) | — | 3 (25%) |

### 5. Filtration Test of Human-Derived Nucleated Cells

Five human nucleated cell lines (see Table 9) were used to test the human-derived nucleated cells capture ability of themodified substrate. The cells were harvested and resuspended with 5 mL PBS, then took 10 μL cells for counting. After counting, the cells were diluted into 1×10⁶ cells/mL and make the final volume of the samples (1×10⁶ cells/mL in PBS)>3 mL. Then, 20 μL, of samples were took and counted three times with LUNA-II Automated Cell Counter (Lows Biosystems). Added 1 mL of sample into the syringe barrel and filtrated through the B-r-1-1 modified substrate. Finally, took 20 μL of the filtrate for cell. counting (3 repeats).

The results show that the modified substrate has a nucleated cell capture rate of at least 99%. This means that when biological samples flow through the modified substrate, most of the human-derived nucleated cells will adhere to the substrate. The results are show in Table 10. The human nucleated cells in the samples are specifically removed after filtered through B-r-H modified substrate.

TABLE 9

| Cell line | Cell type | Growth Properties |
|---|---|---|
| TF1 | bone marrow, erythroblast | suspension |
| Jurkat | peripheral blood, T lymphocyte | suspension |
| PC-3 | prostate, adenocarcinoma | adherent |
| SK-BR-3 | mammary gland/breast, | adherent |
| | adenocarcinoma | |
| K-562 | bone marrow, lymphoblast | suspension |

TABLE 10

| | Before filtration (cells/mL) | | | After filtration (cells/mL) | | |
|---|---|---|---|---|---|---|
| Cell line | 1 | 2 | 3 | 1 | 2 | 3 |
| TF-1 | 9.88 × 10⁵ | 9.88 × 10⁵ | 9.88 × 10³ | 2.50 × 10³ | 0 | 0 |
| Jurkat | 1.05 × 10⁶ | 1.05 × 10⁶ | 1.05 × 10⁶ | 0 | 2.50 × 10³ | 0 |
| PC-3 | 8.12 × 10⁵ | 8.32 × 10⁵ | 9.50 × 10⁵ | 0 | 4.00 × 10³ | 0 |
| SK-BR-3 | 8.24 × 10³ | 8.24 × 10⁵ | 8.16 × 10⁵ | 0 | 0 | 0 |
| K-562 | 1.28 × 10⁶ | 1.37 × 10⁶ | 1.16 × 10⁶ | 0 | 0 | 0 |

What is claimed is:

1. A method for enriching and detecting microorganisms in a biological sample, comprising the following steps:
a) collecting the biological sample including microorganisms and human-derived nucleated cells;
b) filtering the biological sample through a polymer-modified substrate to obtain a filtrate, wherein the human-derived nucleated cells in the biological sample are captured or separated by the polymer-modified substrate and the microorganisms in the biological sample pass or flow through the polymer-modified substrate into the filtrate; and
c) detecting the microorganisms present in the filtrate, wherein:
the human-derived nucleated cells include one or more of erythroblasts, leukocytes and cancer cells; and
the polymer is prepared by the polymerization of one or more monomers having the structure of formula (1): 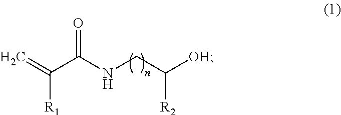
wherein R₁ is independently selected from the group consisting of hydrogen, methyl, ethyl, hydroxyl, C₁₋₁₂ alkyl, phenyl; R₂ is independently selected from the group consisting of hydrogen, methyl, ethyl, C₁₋₆ alkyl, amino, phenyl; and n is an integer of 1 to 5.

2. The method of claim 1, wherein the human-derived nucleated cells are leukocytes, bacteria or fungi.

3. The method of claim 1, wherein a retention rate of the microorganisms in the filtrate is above 65%.

4. The method of claim 3, wherein the retention rate of the microorganisms in the filtrate is above 80%.

5. The method of claim 1, wherein the biological sample further includes erythrocytes, platelets and fibrinogens passing or flowing through the polymer-modified substrate into the filtrate, and all retention rates of the erythrocytes, the platelets and the fibrinogens are above 80%.

6. The method of claim 1, wherein a detection rate of the microorganisms in the filtrate is 2 fold higher than the biological samples without filtration.

7. The method of claim 6, wherein the detection rate of the microorganisms in the filtrate is 40 fold higher than the biological sample without filtration.

8. The method of claim 1, wherein the monomer of formula (1) is N-hydroxyethyl acrylamide, N-(2-hydroxyethyl) acrylamide, NHEMAA, and N-(2-Hydroxyethyl)acrylamide, HEAA.

9. The method of claim 1, wherein the polymer further comprises an additional monomer, which is butyl methacrylate, and the monomer of formula (1) is copolymerized with the additional monomer to form a copolymer.

10. The method of claim 1, wherein the polymer has the structure of formula (2): 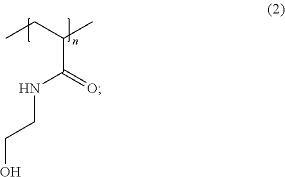
wherein n is an integer of 10 to 50.

11. The method of claim 1, wherein the polymer has the structure of formula (4): 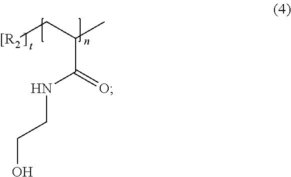
wherein t is an integer of 50 to 90, n is an integer of 10 to 50, and R₂ is

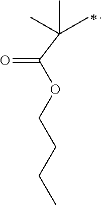

12. The method of claim 1, wherein the polymer is a segmented polymer.

13. The method of claim 1, wherein the polymer is disposed on the substrate by coating, spraying, or impregnating.

14. The method of claim 13, wherein the substrate is polypropylene, polyethylene terephthalate cellulose, polybutylene terephthalate.

15. The method of claim 14, wherein surface elements of the polymer-modified substrate comprise carbon, oxygen, and nitrogen; the total mole percentage of carbon, oxygen, and nitrogen is defined as 100%, the mole percentage of carbon is about 76.22% to 79.84%, the mole percentage of oxygen is about 18.1% to 21.04%, and the mole percentage of nitrogen is about 2.05% to 2.75%.

16. The method of claim 15, wherein the method is used for pathogenic examination of biological samples.

17. The method of claim 16, wherein the biological sample is selected from the group consisting of blood, cerebral spinal fluid, cells, a cellular extract, a tissue sample, and a tissue biopsy.

18. The method of claim 17, wherein the pathogenic examination for biological sample comprises diagnosing sepsis in the individual.

19. The method of claim 1, further comprising:
subjecting the filtrate to DNA purification to obtain a purified DNA; and
analyzing the purified DNA by PCR, qPCR, digital PCR, NGS, MassSpec, or Nanopore sequencing.
